(19) United States
(12) Patent Application Publication
(10) Pub. No.: US 2019/0133918 A1
(43) Pub. Date: May 9, 2019

(54) COSMETIC COMPOSITION COMPRISING AT LEAST ONE SILICONE-POLYURETHANE POLYMER AND A SILICONE RESIN

(71) Applicant: CHANEL PARFUMS BEAUTE, NEUILLY SUR SEINE (FR)

(72) Inventors: Véronique SCHOLLER, VILLEMOMBLE (FR); Chantal MALVEZIN, LAMORLAYE (FR); Catherine RICHARD, BOULOGNE BILLANCOURT (FR); Stéphane MASSON, PANTIN (FR); Hélène DE CLERMONT GALLERANDE, VINCENNES (FR); Elodie CHAMPREDONDE, ORLEANS (FR)

(21) Appl. No.: 16/309,711

(22) PCT Filed: Jun. 13, 2017
(86) PCT No.: PCT/FR2017/051522

§ 371 Date: Dec. 13, 2018

(30) Foreign Application Priority Data
Jun. 14, 2016 (FR) 1655494

(51) Int. Cl. A61K 8/891; A61K 8/898; A61Q 1/06

(52) U.S. Cl. CPC A61K 8/891; A61K 8/898; A61K 8/92; A61K 2800/594; A61Q 1/06

(57) ABSTRACT

Disclosed is a cosmetic make-up or keratin material care composition including at least one silicone-polyurethane polymer and a silicone resin in a physiologically acceptable medium. Also disclosed is a make-up or keratin substance care method involving applying the composition to the keratin material, and the use of an MQ-functional trimethylsiloxy silicate silicone polymer for improving the resistance to rubbing-off and the fastness of a cosmetic composition including at least one silicone-polyurethane polymer in an physiologically acceptable medium.

The object of the present invention is a cosmetic composition for make-up or for caring for keratinous materials comprising at least one silicone-polyurethane polymer and a silicone resin. The object of the invention is also a method for applying makeup or caring for the keratinous materials of human beings involving the application of said composition onto the skin.

Make-up or care compositions are routinely used to provide an aesthetic appearance when applied onto the skin and the lips, and this effect must last a long time. Said compositions must in particular resist various external factors capable of modifying their aesthetic effect, such as sweat or saliva. In particular, cosmetic products, and in particular lipstick, must not migrate or run into wrinkles or small wrinkles or be transferred onto a fabric. They must also be pleasant to apply and the deposition thereof must provide a sensation of comfort to the user while preserving satisfactory aesthetic properties.

It is known, in order to limit the transfer of colour of the cosmetic compositions and improve the stability of their hue, to incorporate polymers called film-forming polymers into the formulas. Examples of film-forming polymers usually used include the polyorganosiloxanes, in particular the polydimethylsiloxanes (PDMS or Dimethicone) such as those described in the documents U.S. Pat. No. 6,780,402 (L'Oréal), U.S. Pat. No. 5,318,775 (Mary Kay Cosmetics), U.S. Pat. No. 4,699,780 (Estee Lauder); and U.S. Pat. No. 4,578,266 (Revlon). More recently, the use of polymers such as silicone-polyurethane has grown.

However, these film-forming polymers do not always allow good results to be obtained in terms of stability of the colour of the make-up and resistance to rubbing.

It would therefore be desirable to have cosmetic compositions having good properties of stability of the make-up, in particular of the colour, while having improved properties of resistance to rubbing.

The goal of the present invention is therefore to propose a composition for make-up or for caring for keratinous materials, in particular the lips, having excellent properties of stability of the make-up, and in particular of the colour thereof, and of resistance to rubbing.

The object of the invention is therefore, according to a first aspect, a cosmetic composition for make-up or for caring for keratinous materials comprising, in a physiologically acceptable medium, at least one silicone-polyurethane polymer and a silicone resin.

The object of the invention is also, according to a second aspect, a method for applying make-up or for caring for keratinous materials, in particular the lips, involving the application of such a composition onto said keratinous materials.

Finally, the object of the invention is, according to a third aspect, the use of a specific silicone resin, in order to improve the resistance to rubbing and the stability of a cosmetic composition comprising, in a physiologically acceptable medium, at least one silicone-polyurethane polymer.

Indeed, the applicant should be given credit for observing that it is possible to formulate compositions having improved properties of stability and resistance to rubbing by associating a specific silicone-polyurethane polymer with a silicone resin, wherein said silicone-polyurethane polymer can be used in a concentration sufficiently low to not alter the resistance to rubbing of to the composition.

### Polymer of Silicone-Polyurethane

The composition according to the invention comprises at least one polymer of silicone-polyurethane. In the broadest sense of the invention, the choice of the polyurethanne silicone polymer does not particularly need to be limited and can cover any polymer comprising organosiloxane units and urethane bonds. According to one embodiment, the polymer of silicone-polyurethane is the product of a reaction of a polyorganosiloxane functionalised by hydroxyl groups, preferably comprising two or more hydroxyl groups, with a diisocyanate compound. The polyorganosiloxane functionalised by hydroxyl groups typically corresponds to the structure having the formula I:

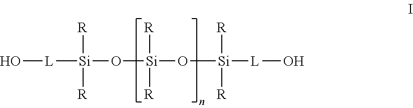

where R is chosen independently at each occurrence from an atom of hydrogen, a hydroxyl group, and optionally substituted hydrocarbon groups containing 1 to 10 atoms of carbon, and in particular from a substituted or non-substituted alkyl, alkenyl, alkynyl, aryl, aryl-alkyl or alkyl-aryl group; preferably, R is chosen from optionally substituted linear, cyclic or branched C1-6 alkyl or alkenyl groups, including, without any limitation, the groups methyl, ethyl, propyl, isopropyl, butyl, isobutyl, t-butyl, amyl, hexyl, cyclohexyl, vinyl, C1-8 allyl or aryl, aryl-alkyl or alkyl-aryl, including, without any limitation, phenyl, benzyl, tolyl, xylyl;

wherein each of the above groups R can be optionally substituted by one or more heteroatoms, including oxygen, nitrogen, phosphorus and a halogen, in particular fluorine, as illustrated by the fluoroalkyl (perfluoroalkyl) groups such as mono-, di- and tri fluoromethyl, perfluorophenyl, and the C1-6 substituted amino alkyl groups, including those corresponding to the formula —(CH₂)₁₋₆—NR^{N}₂ and —(CH₂)₁₋₆—NR^{N}—(CH₂)₁₋₆—NR^{N}₂ where R^{N} is typically hydrogen, but can also be a methyl, ethyl, propyl or equivalent group; polyether groups, including, without any limitation, the polyethylene oxide groups corresponding to the formula —(CH₂CH₂O)ₙ—, the propylene oxide groups corresponding to the formula —(CH(CH₃)CH₂O)ₙ— and combinations of said groups; and amine oxide, phosphate, hydroxyl, ester and/or carboxylate functions or the equivalent; or

wherein R can comprise an additional group—L-OH;

wherein L is either a bond or a linkage group; preferably, L is a linkage group chosen from the divalent hydrocarbons having 1 to 10 atoms of carbon, including a divalent alkyl, alkenyl, alkynyl, aryl, alkyl-aryl or aryl-alkyl group, for example such as a C1-10 alkyl group, including, without any limitation, the bivalent groups having the formula —(CH₂)₁₋₁₀—, preferably —(CH₂)₁₋₆—, and more preferably, L is —CH₂CH₂—CH₂;

and where n is an integer between 0 and 5000, preferably between 1 and 200, more preferably between 10 and 100, and even more preferably between 10 and 50. Preferably, R represents at least one or more occurrences of a methyl group, more preferably, R represents a methyl group in the entirety or quasi-entirety of the occurrences, which means that R represents a methyl group in more than 90% of the occurrences, in particular in more than 95% of the occurrences, or even in more than 98% of the occurrences.

According to one embodiment of the invention, the polyorganosiloxane functionalised by hydroxyl groups comprises a polymethylsiloxane corresponding for example to the structure of formula Ia:

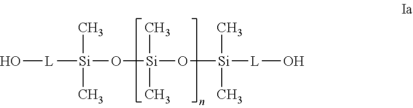

wherein L and n are as defined above.

According to a preferred embodiment of the invention, the polyorganosiloxane functionalised by hydroxyl groups comprises a polymethylsiloxane corresponding for example to the structure of formula Ib:

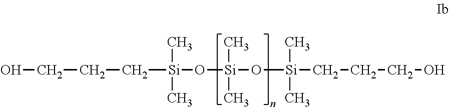

where n is as defined above.

The diisocyanate can in particular correspond to the formula O═C═N—R¹—N═C═O, where R¹ is a divalent hydrocarbon group containing 1 to 20 atoms of carbon, including optionally substituted by one or more heteroatoms, and in particular R¹ can be chosen from a cyclic alkyl group, alkenyl, alkynyl, aryl, alkyl-aryl or linear, cyclic or branched aryl-alkyl, optionally substituted, including, without any limitation:

i. a group having the formula:

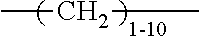

ii. a group having the formula:

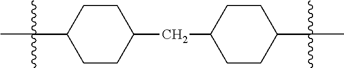 [SMILES: *C1CCC(CC2CCC(*)CC2)CC1]

iii. a group having the formula:

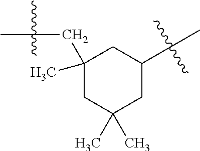 [SMILES: *CC1(C)CC(*)CC(C)(C)C1]

iv. a group having the formula:

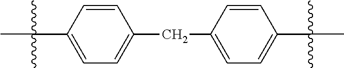 [SMILES: *c1ccc(Cc2ccc(*)cc2)cc1]

and;

v. a group having the formula:

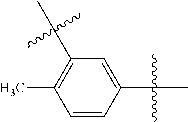 [SMILES: *c1ccc(C)c(*)c1]

and combinations thereof.

The diisocyanates suitable in the context of the invention include, in a non-limiting manner, toluene diisocyanate; methylene diphenyl diisocyanate, including 2,2′-MDI, 2,4′-MDI and 4,4′-MDI; 1,6-hexamethylene diisocyanate; isophorone diisocyanate; methylene dicyclohexyl diisocyanate; xylene diisocyanate; cyclohexane diisocyanate; 3,3′-dimethyl-4,4′-diphenylmethane diisocyanate; p-phenylene diisocyanate; m-phenylene diisocyanate; 4,4′-isopropylidene dicyclohexyl isocyanate; and their equivalents.

In a preferred embodiment, the diisocyanate is chosen from the group consisting of 1,6-hexamethylene diisocyanate, methylene dicyclohexyl diisocyanate, isophorone diisocyanate and the combinations thereof. In one embodiment, the diisocyanate comprises or consists substantially of 1,6-hexamethylene diisocyanate. In another embodiment, the diisocyanate comprises or consists substantially of isophorone diisocyanate. In yet another embodiment, the diisocyanate comprises or consists substantially of methylene dicyclohexyl diisocyanate, this embodiment being particularly preferred.

According to a preferred embodiment, the polymer of silicone-polyurethane according to the invention comprises recurrent units derived from the polyorganosiloxane functionalised by hydroxyl groups and from the diisocyanate in the form of an AB alternating copolymer, where the unit A has the structure having the formula II:

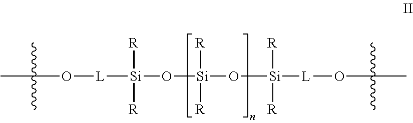

wherein R, L and n are defined as above with respect to the formula I, Ia, Ib and le, and where the unit B has the structure having the formula III:

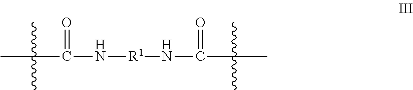

where R′ [symbol] is as defined above, and wherein the units A and B are arranged in a linear, branched or cyclic configuration, preferably linear.

The polymer can also comprise branching points or grafting points in the polyorganosiloxane in which one or more groups R in the formula I or II is a group such that:

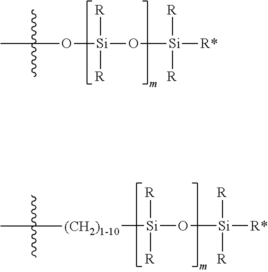

wherein R is as defined for the formula I, and R* can represent an -L-O— group further coupling the lateral chain of a unit B having the formula III, which can in turn be further coupled to the unit A having the formula II, and so on, or R* can represent -L-OH, a group R as defined above, or an end group. When the polyorganosiloxane comprises branching points or grafting points of this type, they can be present in the form of grafts of the T or Q type, where T indicates that only one group R on the Si atom is a polyorganosiloxane chain, as indicated above and Q indicates that both groups R are polyorganosiloxanes. These types of polyorganosiloxane compounds are called copolymer of a polymer of silicone-polyurethane and of a resin T or resin Q, branched or grafted.

The polymers of silicone-polyurethane can also be prepared from functionalised prepolymers of isocyanate. For example, an isocyanate prepolymer can be a difunctional or multifunctional polyorganosiloxane isocyanate, such as the polyorganosiloxane diisocyanate shown below in the formula V:

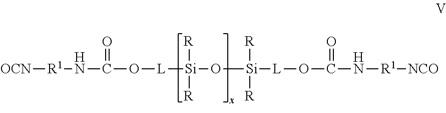

wherein R, R¹ [symbol] and L are as defined above, and where x is an integer between 0 and 5000, preferably between 1 and 200, more preferably between 10 and 100, and even more preferably between 10 and 50. The prepolymer can also be multifunctional by introducing additional isocyanate groups that carry one or more groups R. The prepolymer of functionalised isocyanate is reacted with a polyorganosiloxane functionalised by hydroxyl groups in order to obtain a compound having the formula I or a multifunctional equivalent thereof. The prepolymer according to the formula V has, in general, a molecular weight from 4000 to approximately 15,000 Daltons. The prepolymer according to the formulas I, Ia, Ib and typically has a molecular weight from 250 to approximately 15,000 Daltons.

In one embodiment of the invention, the polymer of silicon-polyurethane does not have or substantially does not have any polyalkylene glycol subunits, in particular polyethylene glycol (PEG) or polypropyleneglycol (PPG). “Substantially does not have any” means that the polymer comprises less than approximately 1% by weight, preferably less than approximately 0.5% by weight, and more preferably less than approximately 0.1% by weight of subunits polyalkyleneglycols.

In a preferred embodiment, the polymer of silicone-polyurethane used in the cosmetic compositions of the invention is a linear polymer comprising the product of a reaction of the formula Ib with a diisocyanate chosen from the group consisting of 1,6-hexamethylene diisocyanate, methylene dicyclohexyl diisocyanate, isophorone diisocyanate and the combinations thereof.

A polymer of silicone-polyurethane is for example available from Siltech Corporation in the form of a premixture in isododecane, under the product reference SILMER UR 5050 or UR 100100.

The composition according to the invention comprises in particular the polymer of silicone-polyurethane in a concentration ranging from 8 to 16%, by weight, of polymer active material, with respect to the total weight of the composition, preferably from 10 to 15% by weight.

Indeed, the applicant should be given credit for observing that compositions comprising more than 16% silicone-polyurethane active material had less resistance to rubbing and stability.

### Silicone Resin

The composition according to the invention, comprises at least one resin itself silicone.

In general, the term “resin” means a compound, the structure of which is three-dimensional. Thus, in the sense of the present invention, a polydimethylsiloxane is not a silicone resin.

The nomenclature of the silicone resins (also called resins of siloxanes) is known under the name “MDTQ”, the resin being described according to the various siloxane monomer units that it comprises, each of the letters “MDTQ” characterising a type of unit.

The letter “M” represents the Monofunctional unit having the formula R1R2R3SiO_{1/2}, the atom of silicon being connected to a single atom of oxygen in the polymer comprising this unit.

The letter “D” means an R1R2SiO_{2/2} Difunctional unit in which the atom of silicon is connected to two atoms of oxygen.

The letter “T” represents a Trifunctional unit having the formula R1SiO_{3/2}.

Such resins are described for example in the patent applications U.S. Pat. Nos. 2,676,182, 3,627,851, 3,772,247, 5,248,739 and also U.S. Pat. Nos. 5,082,706, 5,319,040, 5,302,685 and 4,935,484.

In the patterns M, D, T defined above, R, namely R1 and R2, represents a hydrocarbon radical (in particular alkyl) having 1 to 10 atoms of carbon, a phenyl group, a phenylalkyl group or a hydroxyl group.

Finally, the letter “Q” means an SiO_{4/2} tetrafunctional unit in which the atom of silicon is bound to four atoms of oxygen themselves bound to the rest of the polymer.

Various silicone resins having different properties can be obtained from these various units, the properties of these polymers varying according to the type of monomers (or units), the nature and the number of the radical R, the length of the polymer chain, the branching factor, and the size of the side chains.

Wherein the silicone resins used in the compositions according to the invention can be for example silicon resins of the type MQ, the type T or the type MQT. According to a preferred embodiment, the resins MQ, T and MQT are in solid form, in particular in the form of a powder.

According to a preferred embodiment, the silicone resins used in the compositions according to the invention are film-forming, that is to say that they allow a film to be formed on the keratinous materials onto which they are applied. This excludes in particular the resins of polymethylsilsesquioxane types (or T resins) that are in the form of an insoluble powder and are not film-forming.

According to another preferred embodiment, the silicone resins used in the compositions according to the invention are soluble when hot in silicone solvents, as opposed to the resins of polymethylsilsesquioxane types (or T resins) that are in the form of an insoluble powder.

According to a preferred embodiment, an MQ resin is used. Examples of silicone resins of the MQ type include the alkylsiloxysilicates having the formula [(R1)₃Si_{1/2}]ₓ(SiO_{4/2})_{y} (MQ units) in which x and y are integers from 50 to 80, and such that the group R1 is a radical as defined above, and preferably is an alkyl group having 1 to 8 atoms of carbon or a hydroxyl group, preferably, a methyl group. The MQ resins of the trimethylsiloxysilicate type are thus preferred.

Examples of solid silicone resins of the MQ type of the trimethylsiloxysilicate type include those marketed under the reference SR1000 by the company General Electric, under the reference TMS 803 by the company Wacker, under the name “KF-7312J” by the company Shin-Etsu, “DC 749”, “DC 593” by the company Dow Corning.

Examples of silicone resins comprising siloxysilicate MQ units also include the phenylalkylsiloxysilicate resins, such as phenylpropyldimethylsiloxysilicate (Silshine 151 marketed by the company General Electric). The preparation of such resins is described in particular in the patent U.S. Pat. No. 5,817,302.

Examples of silicone resins of the T type include the polysilsesquioxanes having the formula (RSiO_{3/2})ₓ (T units) in which x is greater than 100 and such that the group R is an alkyl group having 1 to 10 atoms of carbon, wherein said polysilsesquioxanes can further comprise Si—OH end groups. Preferably, the polymethylsilsesquioxane resins in which R represents a methyl group can be used, for example such as those marketed:
by the company Wacker under the reference Resin MK such as Belsil PMS MK: polymer comprising repetitive CH₃SiO_{3/2} units (T units), which can also comprise up to 1% by weight (CH₃)₂SiO_{2/2} units (D units) and having an average molecular weight of approximately 10,000 g/mol, or
by the company SHIN-ETSU under the references KR-220L that are composed 11 of T units having the formula CH₃SiO_{3/2} and have Si—OH end groups (silanol), under the reference KR-242A that comprise 98% T units and 2% D dimethyl units and have Si—OH end groups or under the reference KR-251 comprising 88% T units and 12% D dimethyl units and have Si—OH end groups.

As a resin comprising MQT units, in particular those cited in the document U.S. Pat. No. 5,110,890 are known. A preferred form of resins of the MQT type are the MQT-propyl resins (also called MQTPr). Such resins that can be used in the compositions according to the invention are in particular those described and prepared in the application WO 2005/075542, the contents of which are incorporated here by reference. The MQ-T-propyl resin preferably comprises the following units:

(i) (R1₃SiO_{1/2})ₐ

(ii) (R2₂SiO_{1/2})_{b}

(iii) (R3SiO_{1/2})_{c} and

(iv) (SiO_{4/2})_{d}

with R1, R2 and R3 independently representing a hydrocarbon radical (in particular alkyl) having 1 to 10 atoms of carbon, a phenyl group, a phenylalkyl group, or a hydroxyl group and preferably an alkyl radical having 1 to 8 atoms of carbon or a phenyl group,

a being between 0.05 and 0.5,

b being between zero and 0.3,

c being greater than zero,

d being between 0.05 and 0.6, a+b+c+d=1, and

a, b, c and d being molar fractions, under the condition that more than 40%, in moles, of the groups R3 of the resin of siloxane are propyl groups.

The resins of siloxane that can be used according to the invention can be obtained by a method comprising the reaction of:

A) an MQ resin comprising at least 80%, in moles, of (R1₃SiO_{1/2})ₐ and (SiO_{4/2})_{d} units, R1 representing an alkyl group having 1 to 8 atoms of carbon, an aryl group, a carbinol group or an amino group, a and d being greater than zero, the ratio a/d being between 0.5 and 1.5; and of

B) a resin of propyl T comprising at least 80%, in moles, of (R3SiO_{1/2})_{c} units, R3 representing an alkyl group having 1 to 8 atoms of carbon, an aryl group, a carbinol group or an amino group, c being greater than zero, under the condition that at least 40%, in moles, of the groups R3 are propyl groups, where the mass ratio A/B is between 95:5 and 15:85, preferably the mass ratio A/B is 30:70.

The composition according to the invention comprises in particular a silicone resin, preferably MQ, in a concentration ranging from 1 to 20% resin by weight, with respect to the total weight of the composition, preferably from 5 to 10% by weight.

### Physiologically Acceptable Medium

The composition according to the invention comprises a medium that is physiologically acceptable and preferably cosmetically acceptable, that is to say, which does not have any harmful side effects and in particular which does not produce redness, temperature, pain or tingling unacceptable to a user of cosmetic products.

### Oils

The physiologically acceptable medium can in particular comprise at least one oil.

In the sense of the present invention, “oil” means a compound that is liquid at room temperature (25° C.) and that, when it is introduced at a concentration of at least 1% by weight into water at 25° C., is not at all soluble in the water or soluble at a rate of less than 10% by weight, with respect to the weight of the oil introduced into the water.

The oil can be volatile or non-volatile, polar or apolar. A person skilled in the art will take care to choose the oils forming the physiologically acceptable medium of the composition according to the invention in such a way that said oils are compatible with the acrylate silicone polymer and the resin of silicone that it contains.

Examples of oils that can be used in the compositions according to the invention include in particular hydrocarbon oils, silicone oils, fluorosilicone oils, fluorinated oils, and the mixtures thereof.

Among these oils, it is preferred for the composition according to the invention to comprise at least one hydrocarbon oil.

“Hydrocarbon oil” means an oil containing only atoms of hydrogen and of carbon.

The hydrocarbon oil can be volatile, and in particular have a flash point ranging from 40° C. to 120° C., preferably ranging from 40° C. to 55° C., and preferably ranging from 40° C. to 50° C. The volatile hydrocarbon oil can in particular be chosen from the volatile hydrocarbon oils having 8 to 16 atoms of carbon and the mixtures thereof, and in particular:
the C₈-C₁₆ branched alkanes such as the C₈-C₁₆ iso-alkanes (also called isoparaffins), isododecane, isodecane, isohexadecane, and for example the oils sold under the brand names of Isopars or Permetyls,
the linear alkanes, for example such as the n-dodecane (C12) and the ntetradecane (C14) sold by Sasol under the references PARAFOL 12-97 and PARAFOL 14-97, respectively, and the mixtures thereof, the mixture undecane-tridecane (Cétiol UT), the mixtures of n-undecane (C11) and n-tridecane (C13) obtained in the examples 1 and 2 of the application WO2008/155059 from Société Cognis and the mixtures thereof.

The volatile hydrocarbon oil is preferably isododecane.

The hydrocarbon oil can be non-volatile and hydrocarbon, preferably polar.

In particular, said non-volatile oil can be an ester oil, in particular having between 18 and 70 atoms of carbon.

Examples include the mono-, di-, and tri-esters.

The ester oils can in particular be hydroxylated.

The non-volatile ester oil can preferably be chosen from:
the monoesters comprising between 18 and 40 atoms of carbon in total, in particular the monoesters having the formula R₁COOR₂ in which R1 represents the rest of a linear or branched fatty acid comprising from 4 to 40 atoms of carbon and R2 represents a hydrocarbon chain, in particular branched, containing from 4 to 40 atoms of carbon, under the condition that R1+R2 is 18, for example such as Purcellin oil (cetostearyl octanoate), isononyl isononanoate, C12 to C15 alcohol benzoate, ethyl 2-hexyl palmitate, octyldodecyl neopentanoate, octyl-2 dodecyl stearate, octyl-2 dodecyl erucate, isostearyl isostearate, octyl-2 dodecyl benzoate, octanoates, decanoates or ricinoleates of alcohols or of polyalcohols, isopropyl myristate, isopropyl palmitate, butyl stearate, hexyl laurate, 2-ethyl hexyl palmitate, 2-hexyl-decyl laurate, 2-octyl-decyl palmitate, 2-octyldodecyl myristate, 2-diethyl-hexyl succinate. Preferably, these are esters having the formula R₁COOR₂ in which R1 represents the rest of a linear or branched fatty acid comprising from 4 to 40 atoms of carbon and R2 represents a hydrocarbon chain, in particular branched, containing from 4 to 40 atoms of carbon, R1 and R2 being such that R1+R2 is 18. Preferably, the ester comprises between 18 and 40 atoms of carbon in total. Examples of preferred monoesters include isononyl isononanoate.
the diesters, in particular comprising between 18 and 60 atoms of carbon in total, in particular between 18 and 50 atoms of carbon in total. In particular, the diesters of carboxylic diacid and of monoalcohols, such as preferably diisostearyl malate, can be used. Alternatively, the diesters can be the diesters of glycol and of carboxylic monoacids, such as neopentylglycol diheptanoate or polyglyceryl-2 diisostearate (in particular such as the compound sold under the product reference DERMOL DGDIS by the company Alzo)
the triesters, in particular comprising between 35 and 70 atoms of carbon in total, in particular such as the triesters of carboxylic triacid, such as triisostearyl citrate, or tridecyl trimellitate, or the triesters of glycol and of carboxylic monoacids such as polyglycerol-2 triisostearate;
the tetraesters, in particular having a total number of carbon ranging from 35 to 70, such as the tetraesters of pentaerythritol or of polyglycerol and of a carboxylic monoacid, for example such as pentaerythrityl tetrapelargonate, pentaerythrityl tetraisostearate, pentaerythrityl tetraisononanoate, glyceryl tri decyl-2 tetradecanoate, polyglyceryl-2 tetraisostearate or pentaerythrityl tetra decyl-2 tetradecanoate
the polyesters obtained by condensation of dimer and/or trimer of unsaturated fatty acid and of diol such as those described in the patent application FR 0 853 634, such as in particular dilinoleic acid and 1,4-butanediol.
the esters and polyesters of dimer diol and of mono- or dicarboxylic acid, such as the esters of dimer diol and of fatty acid and the esters of dimer diols and of carboxylic diacid dimer, in particular that can be obtained from a carboxylic diacid dimer derived in particular from the dimerisation of an unsaturated fatty acid in particular a C8 to C34 unsaturated fatty acid, in particular C12 to C22, in particular C16 to C20, and more particularly C18, such as the esters of dilinoleic diacids and of dilinoleic dimer diols, for example such as those marketed by the company NIPPON FINE CHEMICAL under the brand name LUSPLAN DD-DA5® and DD-DA7®
the vinylpyrrolidone/1-hexadecene copolymers, for example such as that sold under the name ANTARON V-216 (also called Ganex V216) by the company ISP (MW=7300 g/mol),—the vegetable hydrocarbon oils such as the triglycerides of fatty acids (liquid at room temperature), in particular of fatty acids having 7 to 40 atoms of carbon, such as the triglycerides of heptanoic or octanoic acid or jojoba oil, in particular, examples include the saturated triglycerides such as caprylic/capric triglyceride, glyceryl triheptanoate, glycerine trioctanoate, the triglycerides of C18_36 acid such as those marketed under the reference DUB TGI 24 marketed by Stéarineries Dubois), and the unsaturated triglycerides such as ricin oil, olive oil, ximenia oil, pracaxi oil;
and the mixtures thereof.

“Silicone oil” means an oil comprising at least one atom of silicon, and in particular at least one Si—O group. The silicone oil can be volatile or non-volatile.

Examples of non-volatile silicone oil include in particular the polydimethylsiloxanes containing at least 8 atoms of silicon, the polyalkylmethylsiloxane, the alkyl chain of which contains 8 to 20 atoms of carbon, and the oils identified by the INCI name phenyl trimethicone.

Examples of volatile silicone oil include in particular certain dimethicones having a viscosity of 5 and 6 cSt, octamethyl cyclotetrasiloxane, decamethyl cyclopentasiloxane, dodecamethyl cyclohexasiloxane, heptamethyl hexyltrisiloxane, heptamethyloctyl trisiloxane, hexamethyl disiloxane, octamethyl trisiloxane, decamethyl tetrasiloxane, dodecamethyl pentasiloxane, the compounds identified by the INCI names methyl trimethicone and caprylyl methicone and the mixtures thereof.

Examples of vegetable oils include in particular the oils of wheat germ, sunflower, grapeseed, sesame, corn, apricot, castor, shea, avocado, olive, soybean, the oil of sweet almonds, palm, colza, cottonseed, hazelnut, macadamia, jojoba, alfalfa, poppyseed, squash, sesame, pumpkin, colza, black currant, evening primrose, millet, barley, quinoa, rye, safflower, candlenut, passion flower, rose hip seed or camellia.

“Fluorinated oils” means an oil containing at least one atom of fluorine, such as nonafluoromethoxybutane or perfluoromethyl-cyclopentane, perfluorodimethylcyclohexane, perfluoroperhydrophenanthrene, perfluorodecalin, and the mixtures thereof, without this list being limiting.

The composition according to the invention preferably comprises 40 to 80% oil by weight, in particular a hydrocarbon oil, in particular 45 to 75% by weight, and more preferably 50 to 70% oil by weight.

### Wax and Lipophilic Gelling Agent

The composition according to the invention can further comprise at least one wax and/or at least one lipophilic gelling agent.

“Wax” means a fatty body having a melting temperature higher than 30° C. and generally lower than 100° C., which is liquid under the conditions of preparation of the composition and has, in the solid state, an anisotropic crystalline structure. Examples of waxes include in particular the vegetable, mineral and synthetic waxes, wherein the latter can advantageously be hydrocarbon or silicone waxes. Mention can also be made of the waxes of Carnauba, Candelilla, rice, bees (Cera alba), optionally functionalised polyethylene, and paraffin, as well as ozokerite, the microcrystalline waxes, the C₁₄₋C₂₂ linear fatty alcohols and the C₈-C₂₀ triesters of acids and of glycerine such as glycerine tribehenate, and the mixtures thereof, without this list being limiting. Mention can also be made of the glycol acetyl stearate marketed by the company VEVY under the brand name CETACENE®.

According to a preferred embodiment, the composition according to the invention comprises at least one apolar wax. Apolar wax means a hydrocarbon or silicone apolar wax.

According to one embodiment, the apolar hydrocarbon wax contains at least 95%, by weight, of chemical compounds consisting of carbon and of hydrogen. These chemical compounds are advantageously chosen from the saturated alkanes, linear or branched. In particular, the apolar wax can be chosen from the linear hydrocarbon waxes. The linear hydrocarbon waxes include the polymers and copolymers of ethylene, the linear paraffin waxes and the Fischer Tropsch waxes.

The apolar wax can alternatively be a wax of the polyoxyalkylene silicone type that is to say a silicone comprising at least one oxyalkylene group of the type (—CxH₂xO)a in which x can vary from 2 to 6 and a is greater than or equal to 2.

According to a preferred embodiment, the composition according to the invention comprises at least one polyethylene wax.

The composition according to the invention can in particular comprise a wax in a concentration ranging from 1 to 20% by weight, preferably from 5 to 15% by weight, with respect to the total weight of the composition.

Examples of lipophilic gelling agents are in particular fillers allowing the rheology or the texture of the composition to be modified.

“Filler” should be understood as the colourless or white particles, mineral or synthetic, solids of any shape, that are in a form that is insoluble and dispersed in the medium of the composition, regardless of the temperature at which the composition is manufactured. The fillers can be mineral or organic of any shape, plates, spherical or oblong, regardless of the crystallographic shape (for example flakes, cubic, hexagonal, orthorhombic, etc.).

In particular, the filler can be chosen from talc, mica, silica, kaolin, a hectorite clay (bentone), the particles of pyrogenic silica, optionally treated hydrophilically or hydrophobically, the powders of polyamide (Nylon®) (Orgasol® from Atochem), of poly-(3-alanine and of polyethylene, the powders of tetrafluoroethylene polymers (Teflon®), lauroyl-lysine, starch, boron nitrate, the polymer hollow microspheres such as those of polyvinylidene/acrylonitrile chloride such as Expancel® (Nobel Industrie), of copolymers of acrylic acid (Polytrap® from the company Dow Corning), the microbeads of silicone resin (Tospearls® from TOSHIBA, for example), precipitated calcium carbonate, the carbonate and hydro-carbonate of magnesium, hydroxyapatite, the hollow microspheres of silica (Silica Beads® from Maprecos), the particles of elastomer polyorganosiloxanes, the metal soaps derived from carboxylic organic acids having 8 to 22 atoms of carbon, preferably 12 to 18 atoms of carbon, the microcapsules of glass or of ceramic, the metal soaps derived from carboxylic organic acids having 8 to 22 atoms of carbon, preferably 12 to 18 atoms of carbon, for example the stearate of zinc, of magnesium or of lithium, zinc laurate, magnesium myristate, and the mixture thereof. Preferably, the filler is chosen from silica, kaolin, bentone, starch, lauroyl-lysine, the particles of pyrogenic silica, optionally treated hydrophilically or hydrophobically, and the mixtures thereof. According to a preferred embodiment, the lipophilic gelling agent is preferably bentone.

The composition used according to the invention can comprise one or more lipophilic gelling agents in a concentration ranging from 0.1 to 13% by weight with respect to the total weight of the composition, in particular from 0.2 to 10% by weight with respect to the total weight of the composition.

### Colouring Materials

The composition according to the invention can further comprise at least one colouring material that can be chosen from pigments, nacres, fat-soluble colouring agents, lacquers (organic pigment) and the mixtures thereof.

Pigments should be understood as white or coloured particles, mineral or organic, insoluble in an aqueous solution, intended to colour and/or opacify the composition.

Examples of mineral pigments that can be used in the invention include the oxides of titanium, zirconium or cerium, as well as the oxides of zinc, iron or chromium, ferric blue, manganese violet, ultramarine, and chromium hydrate.

Examples of organic pigments that can be used in the invention include carbon black, the pigments of type D & C, the lacquers containing cochineal carmine, barium, strontium, calcium, aluminium or the diketopyrrolopyrroles (DPP) described in the documents EP-A-542669, EP-A-787730, EP-A-787731 and WO-A-96/08537.

The pigments used in the cosmetic composition according to the invention can be surface treated with a hydrophobic treatment agent.

The hydrophobic treatment agent can be chosen from the silicones such as the methicones, the dimethicones, the perfluoroalkylsilanes, the alkyl alkoxy silanes; the fatty acids such as stearic acid; the metal soaps such as aluminium dimyristate, the aluminium salt of hydrogenated tallow glutamate, the perfluoroalkyl phosphates, the perfluoroalkyl silanes, the perfluoroalkyl silazanes, the polyoxides of hexafluoropropylene, the polyorganosiloxanes comprising perfluoroalkyl perfluoropolyether groups, the amino acids; the N-acyl amino acids or the salts thereof; lecithin, isopropyl trisostearyl titanate, and the mixtures thereof.

According to a preferred embodiment, the hydrophobic treatment agent is chosen from the alkyl alkoxy silanes, in particular octyl triethoxy silane (OTS).

“Nacres” should be understood as coloured particles of any shape, iridescent or not, in particular produced by certain molluscs in their shell or synthesised and which have an effect of colour via optical interference.

“Colouring agents” should be understood as generally organic compounds soluble in fatty bodies such as oils.

The fat-soluble colouring agents are for example Sudan red, DC Red 17, DC Green 6, β-carotene, Sudan brown, DC Yellow 11, DC Violet 2, DC orange 5, quinoline yellow.

The colouring materials can be present in a concentration ranging from 0.1 to 15% by weight, in particular 1 to 10% by weight, and in particular 1 to 10% by weight, with respect to the total weight of the cosmetic composition.

### Active Ingredients

The composition according to the invention can also contain one or more active ingredients, in particular chosen from the hydrating agents (in particular vitamin E), the scarring agents and/or the anti-ageing agents, of the skin and/or of the lips, and in particular of the lips.

According to this embodiment, the invention also relates to a method for care for and/or applying make-up (non-therapeutic) onto the skin and/or the lips, and in particular the lips comprising the application of a composition according to the invention onto the skin and or the lips.

The deposit created with a composition according to the invention having a good level of stability, allows the active ingredient to be preserved on the skin and/or the lips and thus the effectiveness of the care (hydrating, scarring and/or anti-ageing effect) for the skin and/or the lips to be improved.

The composition used according to the invention can further contain humectants such as hyaluronic acid and the salts thereof and/or the polyols such as glycerine.

### Additional Routine Cosmetic Ingredients

The composition according to the invention can also comprise any routine cosmetic ingredient that can be chosen in particular from antioxidants, perfumes, preservatives, neutralisers, surfactants, solar filters, vitamins, hydrating agents, self-tanning compounds, anti-wrinkling active ingredients, emollients, hydrophilic or lipophilic active ingredients, anti-free-radical agents, deodorising agents, sequestering agents, and the mixtures thereof.

In particular, the composition according to the invention can comprise at least one solar filter.

### Galenic

The composition according to the invention can be advantageously used for care for or for applying make-up onto the skin or the lips, and in particular for applying make-up onto the lips.

In particular, the product of the invention can be in the form of a base layer in the context of a product for dual-layer make-up for the skin or lips.

### Method for Applying Make-Up

The object of the invention is also a method for applying make-up onto or for caring for keratinous materials, in particular the lips, comprising the application, onto said keratinous materials, of at least one composition as defined above.

According to a preferred embodiment, the method according to the invention comprises:
the application, onto said keratinous materials, of at least one composition as defined above, as a base composition (also called “base coat”), and
the application, onto said base composition, of a coating composition (also called “top coat”).

The “top coat” coating composition is in particular chosen by a person skilled in the art in such a way as to be incompatible with the base composition in order to prevent the migration of the components of each composition to the other, which would alter the stability and the intensity of the colour provided by the base composition according to the invention, and would reduce the shine provided by the “top coat” coating composition.

The coating composition (“top coat”) comprises, preferably, at least one non-volatile oil, preferably shiny, incompatible with the base composition. The non-volatile oil can in particular be a non-volatile silicone oil.

The coating composition (“top coat”) allows shine to be provided to the base composition, and the sensation of stickiness provided by the base composition when it is applied alone to be reduced or even eliminated.

### Use

The object of the invention is also, according to another aspect, the use of a specific silicone resin, in order to improve the resistance to rubbing and the stability of a cosmetic composition comprising, in a physiologically acceptable medium, at least one silicone-polyurethane polymer.

The following examples are given as illustrations and are non-limiting to the present invention. The percentages are weight percentages.

### EXAMPLES

Two liquid formulas for the lips were prepared, having the following compositions:

| | Composition 1 according | Comparative |
|---|---|---|
| | to the invention (% by | composition |
| | weight) | 2 (% by weight) |
| MQ silicone resin (Belsil | 7.0 | — |
| TMS 803 from Wacker) | | |
| Silicone-polyurethane | 30.0 | 30.0 |
| polymer at 40% in | | |
| isododecane (SILMER | | |
| UR-5050 from Siltech) | | |
| Isododecane | 27.0 | 34.0 |
| Polyethylene wax | 10.0 | 10.0 |
| Hectorite (Bentone Gel) | 10.0 | 10.0 |
| Mica | 7.5 | 7.5 |
| Colouring materials | 6.0 | 6.0 |
| Diisostearyl malate | 1.5 | 1.5 |
| Vitamin E | 0.5 | 0.5 |
| Preservatives | 0.5 | 0.5 |

The formulas were applied (3 successive layers) onto the lips of a sample group of 9 people.

The properties of the compositions were evaluated visually by an experienced make-up artist on the basis of the following criteria:
stability of the colour of the compositions after a meal (pasta with sauce),
resistance to rubbing (wiping of the compositions with a tissue).

The make-up artist thus observed that the composition 1 according to the invention left more residual deposit on the lips after wiping with a tissue than the comparative composition 2.

Moreover, the make-up removal of the composition 1 was much more difficult than that of the comparative composition 2 since the composition 1 according to the invention adheres more to the mucosa (better stability of the make-up).

In another trial, the formulas of the compositions 1 and 2 were again applied (3 successive layers) onto the lips of a sample group of 9 people, and then, this time, coated with a colourless shiny “top coat” having the following formula:

| | Colourless top coat (% by weight) |
|---|---|
| Non-volatile silicone oil | 25.0 |
| Viscosity 1,000,000 cs | |
| Non-volatile silicone oil | 75.0 |
| Viscosity 350 cs | |

The make-up result was evaluated visually by an experienced make-up artist on the basis of the following criteria:
hue,
homogeneity,
the sharpness of the contours,
shine.

The make-up artist thus observed that composition 1 according to the invention allowed a hue to be obtained that was more intense (in particular throughout the day), was homogenous during application, and ran less (sharper contours) than the comparative composition 2.

A test of stability of the compositions after a meal (pasta with sauce) also demonstrated better stability of the hue obtained with composition 1 according to the invention than with comparative composition 2.

1. Cosmetic composition for make-up or for caring for keratinous materials comprising, in a physiologically acceptable medium, at least one silicone-polyurethane polymer and a silicone resin.

2. Composition according to claim 1, wherein the silicone-polyurethane polymer is the product of a reaction of a polyorganosiloxane functionalised by hydroxyl groups with a diisocyanate compound.

3. Composition according to claim 2, wherein the polyorganosiloxane functionalised by hydroxyl groups corresponds to the structure having the formula Ib: 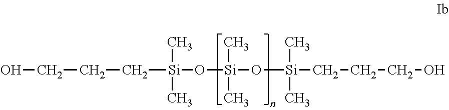
wherein n is an integer between 0 and 5000, preferably between 1 and 200, more preferably between 10 and 100, and even more preferably between 10 and 50.

4. Composition according to claim 2, wherein the diisocyanate compound corresponds to the formula O═C═N—R¹—N═C═O, where R¹ is a divalent hydrocarbon group containing 1 to 20 atoms of carbon, and in particular R¹ is a group having the formula:

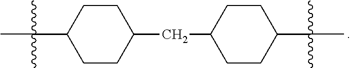 [SMILES: *C1CCC(CC2CCC(*)CC2)CC1]

5. Composition according to claim 4, wherein the diisocyanate compound is chosen from the group consisting of 1,6-hexamethylene diisocyanate, methylene dicyclohexyl diisocyanate, isophorone diisocyanate and the combinations thereof.

6. Composition according to claim 1, wherein the polymer of silicone-polyurethane is present in a concentration ranging from 8 to 16%, by weight, of polymer active material, with respect to the total weight of the composition.

7. Composition according to claim 1, wherein the silicone resin is a resin of the type MQ, the type T or the type MQT.

8. Composition according to claim 1, wherein the silicone resin is a resin of the type MQ alkylsiloxysilicates having the formula [(R1)₃Si_{1/2}]ₓ(SiO_{4/2})_{y} in which x and y are integers ranging from 50 to 80, and the group R1 is a hydrocarbon radical having 1 to 10 atoms of carbon, a phenyl group, a phenylalkyl group or a hydroxyl group.

9. Composition according to the claim 8, wherein the MQ resin of the trimethylsiloxysilicate type.

10. Composition according to claim 1, wherein the silicone resin is present in a concentration ranging from 1 to 20% resin by weight, with respect to the total weight of the composition.

11. Composition according to claim 1, wherein the physiologically acceptable medium comprises at least one volatile or non-volatile hydrocarbon oil.

12. Composition according to claim 11, wherein the volatile hydrocarbon oil comprises 8 to 16 atoms of carbon.

13. Composition according to claim 1, further comprising at least one wax and/or at least one lipophilic gelling agent.

14. Composition according to claim 13, wherein the wax is a polyethylene wax and the lipophilic gelling agent is a clay.

15. Composition according to claim 1, further comprising at least one colouring material chosen from pigments, nacres, fat-soluble colouring agents, lacquers (organic pigment) and the mixtures thereof.

16. Method for applying make-up or for caring for keratinous materials, involving the application of at least one composition according to claim 1 onto said keratinous materials.

17. Method for applying make-up or for caring for keratinous materials, comprising:
the application, onto said keratinous materials, of at least one composition according to claim 1 as a base composition, and
the application, onto said base composition, of a coating composition.

18. Method according to claim 17, wherein the coating composition is incompatible with the base composition.

19. (canceled)

20. Composition according to claim 2, wherein the polyorganosiloxane functionalised by hydroxyl groups corresponds to the structure having the formula Ib: 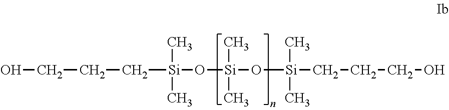
wherein n is an integer between 10 and 50.

21. Composition according to claim 1, wherein the polymer of silicone-polyurethane is present in a concentration ranging from 10 to 15%, by weight, of polymer active material, with respect to the total weight of the composition.
